(19) United States
(12) Patent Application Publication
(10) Pub. No.: US 2017/0281012 A1
(43) Pub. Date: Oct. 5, 2017

(54) OPPORTUNISTIC MEASUREMENTS AND PROCESSING OF USER'S CONTEXT

(71) Applicant: Intel Corporation, Santa Clara, CA (US)

(72) Inventors: Ray Kacelenga, Forest Grove, OR (US); Rajasekaran Andiappan, Ekenas (FI); Uttam K. Sengupta, Portland, OR (US)

(21) Appl. No.: 15/084,344

(22) Filed: Mar. 29, 2016

(51) Int. Cl. A61B 5/0205; A61B 5/1455; A61B 5/0452; A61B 5/0408; A61B 5/00

(52) U.S. Cl. CPC A61B 5/0205; A61B 5/0408; A61B 5/7221; A61B 5/6813; A61B 5/7271; A61B 5/0452; A61B 5/14551; A61B 5/02416

(57) ABSTRACT

Embodiments of the present disclosure provide for an apparatus for opportunistic measurements and processing of a user's context. In one instance, the apparatus may include a processing block, a first sensor having first and second electrodes disposed on a work surface of the apparatus, to provide first readings of a user's physiological context in response to a contact between the electrodes and respective hands of a user, and a second sensor coupled with the processing block and having a sensitive surface embedded in one of the first or second electrode. The second sensor may provide second readings of the user's physiological context and a wake-up signal to the processing block in response to proximity of one of the hands to the sensitive surface. The processing block may facilitate process the user's physiological context in response to a receipt of the wake-up signal. Other embodiments may be described and/or claimed.

### FIELD

Embodiments of the present disclosure generally relate to the field of sensor devices, and more particularly, to providing opportunistic measurements of a user's physiological context.

### BACKGROUND

Today's computing devices may provide for sensing and rendering to a user some user context parameters, such as the user's movements, ambient light, ambient temperature, and the like. The user context parameters may be provided by adding relevant sensors and corresponding logic to a user's computing device. However, the existing methods for provision of the user's context, such as parameters related to the user's state of health, may involve continuous sensor readings and corresponding data processing, which may consume substantial energy, hardware, and computing resources. For example, for a computing device with embedded electrocardiogram (ECG) sensor, once the ECG sensor is turned on, it may run continuously. The output data may or may not be valid ECG, depending on the user's actions with respect to the ECG sensor electrodes. Further, a processing component of a computing device may have to be run (and powered on) continuously, as opposed to on demand, in order to process the ECG data provided by the ECG sensor.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example and not by way of limitation in the figures of the accompanying drawings.

FIG. 1 is a block diagram illustrating an example apparatus for opportunistic measurements of user's physiological context, incorporated with the teachings of the present disclosure, in accordance with some embodiments.

FIG. 2 illustrates examples of disposition of sensors on work surfaces of computing devices, to enable measurements of a user's physiological context, in accordance with some embodiments.

FIG. 3 is a process flow diagram for opportunistic measurements and processing of a user's physiological context, in accordance with some embodiments

FIG. 4 illustrates example graphs of ECG data, in accordance with some embodiments.

FIG. 5 illustrates an example embodiment of an apparatus for opportunistic measurements of a user's physiological context, suitable for use with various components of FIG. 1, in accordance with some embodiments.

FIG. 6 is a process flow diagram for opportunistic measurements and processing of a user's physiological context by the apparatus configured as described in reference to FIGS. 1 and 5, in accordance with some embodiments.

FIG. 7 is a block diagram illustrating an example apparatus for opportunistic measurements of a user's physiological context, in accordance with some embodiments.

FIG. 8 is a process flow diagram for opportunistic measurements and processing of a user's physiological context with an apparatus of FIG. 7, in accordance with some embodiments.

### DETAILED DESCRIPTION

Embodiments of the present disclosure include techniques and configurations for opportunistic measurements of a user's physiological context. Opportunistic measurements may include measurements of the user's context during the user's interaction with an apparatus, e.g., when portions of the user's upper limbs (e.g., hands, palms, fingers, and/or wrists) are disposed on the work surface of the apparatus.

In accordance with embodiments, the apparatus may include a processing block and a first sensor coupled with the processing block having first and second electrodes disposed on a work surface of the apparatus, to provide first readings of a user's physiological context in response to a contact between the first and second electrodes and respective hands of a user during interaction of the user with the apparatus. The apparatus may further include a second sensor coupled with the processing block and having a sensitive surface embedded in one of the first or second electrode of the first sensor. The second sensor may provide second readings of the user's physiological context and further provide a wake-up signal to the processing block in response to at least proximity of a portion of one of the first or second hands to the sensitive surface. The processing block may facilitate processing of the user's physiological context in response to a receipt of the wake-up signal.

In the following detailed description, reference is made to the accompanying drawings that form a part hereof, wherein like numerals designate like parts throughout, and in which are shown by way of illustration embodiments in which the subject matter of the present disclosure may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

The description may use perspective-based descriptions such as top/bottom, in/out, over/under, and the like. Such descriptions are merely used to facilitate the discussion and are not intended to restrict the application of embodiments described herein to any particular orientation.

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “coupled with,” along with its derivatives, may be used herein. “Coupled” may mean one or more of the following. “Coupled” may mean that two or more elements are in direct physical, electrical, or optical contact. However, “coupled” may also mean that two or more elements indirectly contact each other, but yet still cooperate or interact with each other, and may mean that one or more other elements are coupled or connected between the elements that are said to be coupled with each other. The term “directly coupled” may mean that two or more elements are in direct contact.

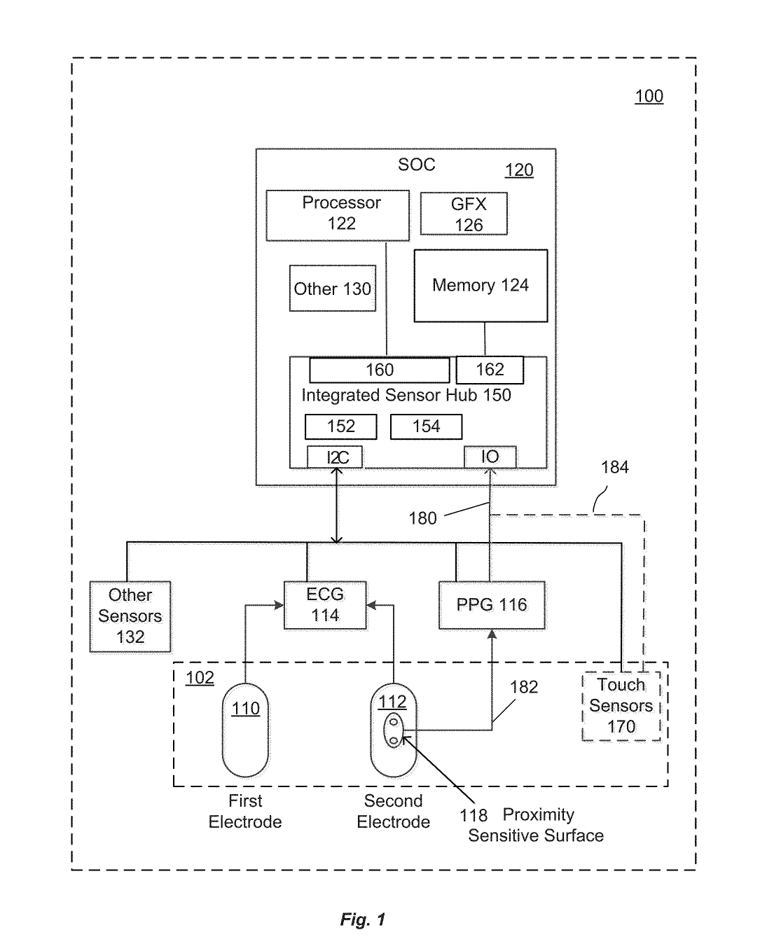

FIG. 1 is a block diagram illustrating an example apparatus 100 for opportunistic measurements of a user's physiological context, incorporated with the teachings of the present disclosure, in accordance with some embodiments. In embodiments, the apparatus 100 may comprise a laptop computer, a desktop computer, a tablet computer, a smartphone, a set-top box, a game controller, a 2-in-1 computing device, or a wearable device.

The apparatus 100 may be configured, for example, to include a sensor arrangement (e.g., a pair of electrodes coupled with a sensor) to provide electrocardiogram (ECG) readings, to measure the natural electrical activity of the heart when the heart is pumping blood to the lungs and the rest of the user's body. The apparatus 100 may be further configured to include another sensor arrangement (e.g., an optical sensor with a touch-sensitive surface) to provide photoplethysmographic (PPG) readings, such as modulation of the blood vessels when the blood volume increases and decreases during cardiac cycles. The modulation information may be used to calculate heart and/or respiration rate, and peripheral oxygen saturation (SpO2) levels in the blood.

Conventional ECG sensors are configured to provide ECG readings only when the ECG electrodes disposed on the apparatus are touched by the user's respective hands (e.g., fingers, palms, or wrists). The ECG sensors may not be configured to provide a wake-up signal to compel a processing unit of the apparatus to begin processing the ECG readings when the circuit formed by the ECG electrodes is closed by the user's respective hands (e.g., fingers, palms, or wrists). Instead, the ECG sensor may be turned on once (e.g., when the apparatus is powered on) and then left to run continuously, thus providing continuous ECG output for processing. The resulting ECG readings may or may not be valid, depending on whether the electrode loop is closed by the user's respective hands.

In embodiments described herein, at least one of the ECG electrodes may include an embedded touch-sensitive surface of the PPG sensor. A wake-up signal may be generated by a conventional PPG sensor in response to a contact with at least one of the user's hands with the touch-sensitive surface. The wake-up signal may be provided to the processing unit of the apparatus, in order to facilitate independent, and in some embodiments simultaneous, processing of the ECG and PPG readings provided by the ECG and PPG sensor arrangements. The embodiments described herein may enable opportunistic measurements of the user's physiological context, while saving processing power of the processing unit of the apparatus.

Referring to FIG. 1, the apparatus 100 may comprise a work surface 102, e.g., a portion of a keyboard (such as a plane in front of the keyboard, or keyboard keys), a bezel, a back side, or other surface that may be accessed by the portions of a user's hands (e.g., hands, palms, finger, or wrists) when interacting with the apparatus 100.

Two or more electrodes (e.g., 110, 112) may be placed in specific areas of the apparatus 100, and may be used to directly measure the heart's electrical activity and heartbeat rate of the user, to sense ECG bio-potentials from left and right hands, palms, or wrists of the user, and provide ECG readings. More specifically, electrodes 110, 112 may be placed on the work surface 102 of the apparatus 100 such as to be positioned on opposite sides of, and distant from, the user's heart.

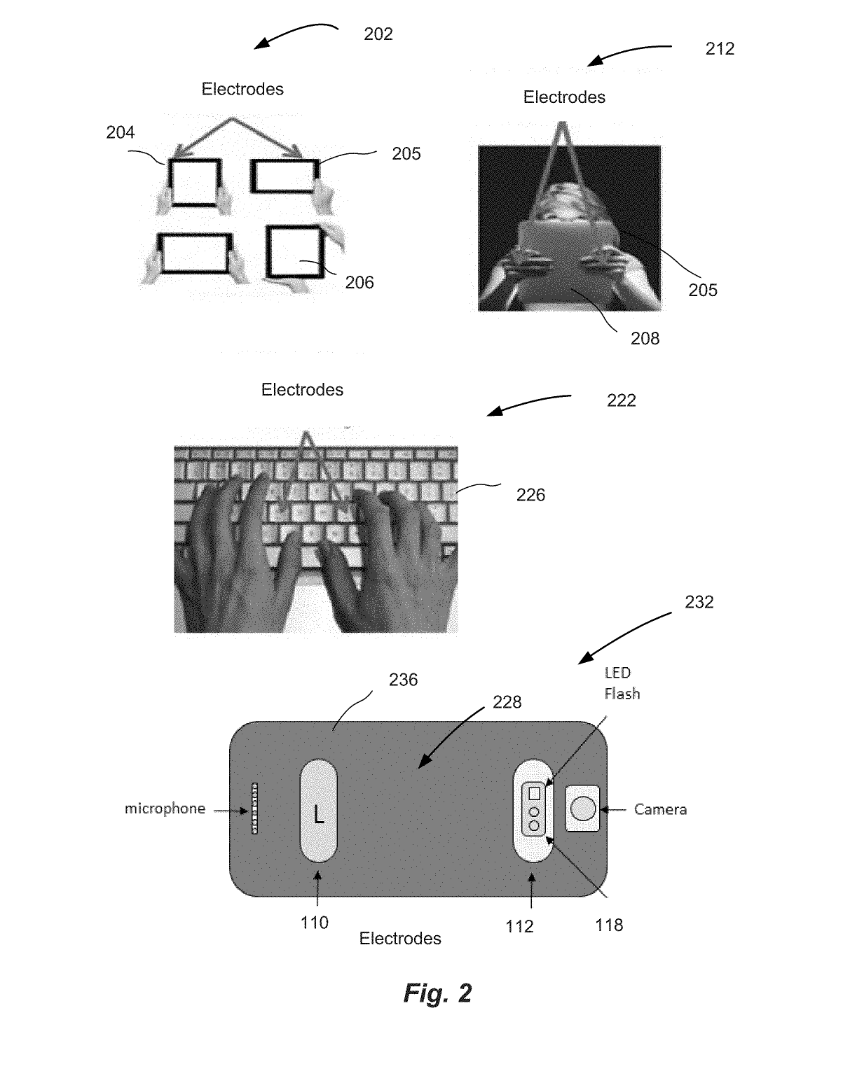

In some embodiments, two or more electrodes 110, 112 may be disposed on the work surface 102 to directly or indirectly (e.g., when the sensors are covered by, or placed behind, an enclosure of the apparatus 100) contact the user's hands, fingers, palms, or wrists 106, when the user's hands, palms, fingers, or wrists are disposed on the work surface 102 to interact with the apparatus 100. For example, electrodes 110, 112 may be placed on the keyboard of a laptop or desktop computer or on the bezel or back side of a tablet computer or a smartphone in positions where users rest their fingers and palms or wrists naturally. Different embodiments of disposition of electrodes 110, 112 on work surfaces of various devices will be described in reference to FIG. 2.

The electrodes 110, 112 may be coupled with a sensor 114 (e.g., ECG sensor), to obtain readings of a user's physiological context in response to a contact between the electrodes 110, 112 and at least portions of respective first and second hands of a user (not shown) during interaction of the user with the apparatus 100.

In general, different types of sensors providing readings of the user's context may be disposed in the apparatus 100 to provide readings related to various user body functions and current physiological state. For example, the sensor 114 may be an ECG sensor and the electrodes 110, 112 may be configured to detect bio-potentials from the user's hands, (e.g., fingers, palms, or wrists) in response to contact with user's hands, in order for the ECG sensor to provide ECG readings, to measure the natural electrical activity of the heart when the heart is pumping blood to the lungs and the rest of the user's body.

In embodiments, the apparatus 100 may include a sensor 116 having a sensitive surface 118, such as touch-sensitive or proximity-sensitive surface that may be embedded in one of the electrodes, such as electrode 112, as shown. In some embodiments, the proximity-sensitive surface 118 may comprise a proximity sensor responsive to proximity of a portion of the user's hand, e.g., a finger. In some embodiments, the sensitive surface 118 may comprise a different type of sensitive surface, e.g., capacitive surface or other sensitive surface. For brevity, the sensitive surface 118 is called hereinafter a proximity-sensitive surface.

In embodiments, the sensor 116 may comprise a PPG sensor. For example, the sensor 116 with the proximity-sensitive surface 118 may comprise an optical sensor to provide PPG readings of the user's physiological context. In embodiments, the sensor 116 with proximity-sensitive surface 118 may comprise a combination of photodetectors and light-emitting diodes (LED) configured to detect a flow of blood, e.g., to user's finger or palm placed on or near the proximity-sensitive surface 118. More specifically, the sensor 116 (PPG sensor) may be configured to measure the absorption and reflection of the radiated light by the volume of the blood in the blood vessels and capture the modulation of the blood vessels when the blood volume increases and decreases during cardiac cycles.

In embodiments, the sensors 114, 116 may be coupled with a processing block 120, such as a physical processor block. In embodiments, the processing block 120 may be integrated in a System-on-a-Chip (SoC) configuration. The processing block 120 may be configured to process readings of the user's physiological context provided by the sensors 114, 116. In embodiments, the processing block 120 may begin processing readings of the user's physiological context provided by the sensors 114, 116 (e.g., ECG and PPG sensors respectively) in response to a receipt of the wake-up (e.g., “Interrupt”) signal 180. In embodiments, the processing block 120 may include an integrated sensor hub 150 having a processor 152 and memory 154, configured to run independently of 120 to process the sensor samples. The wake-up signal 180 may be triggered by a signal 182 generated by the proximity-sensitive surface 118 of the sensor 116, embedded in the electrode 112 as described above. The signal 182 may indicate a contact between at least a portion of one of the hands of the user (e.g., a finger) and the proximity-sensitive surface 118.

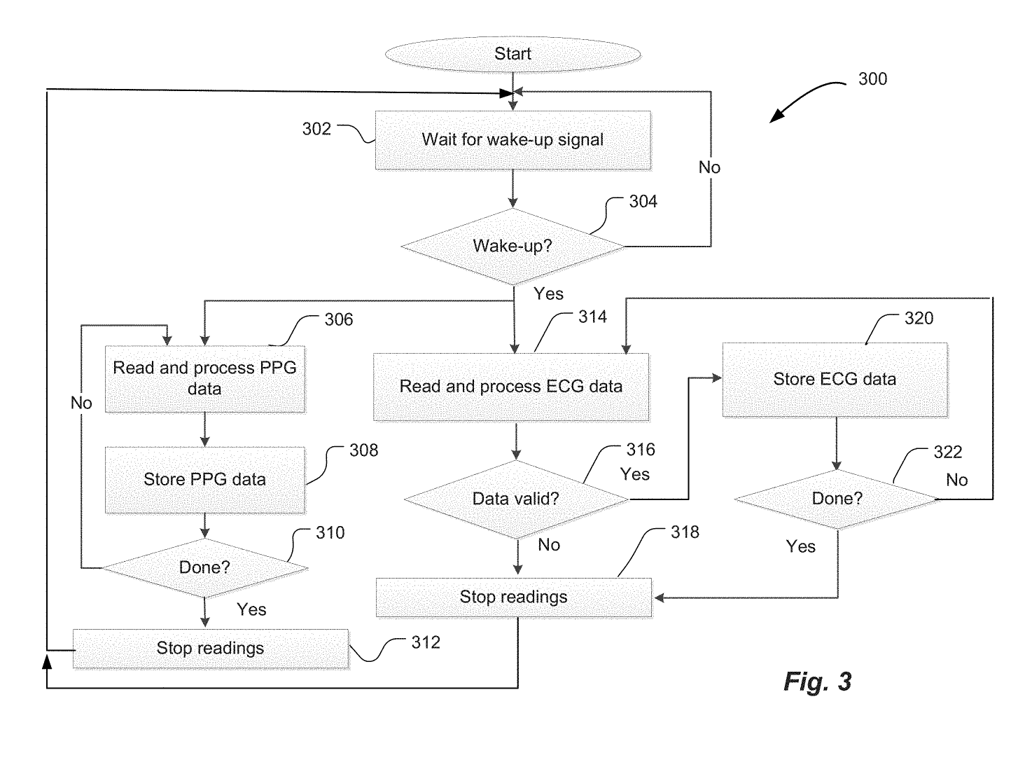

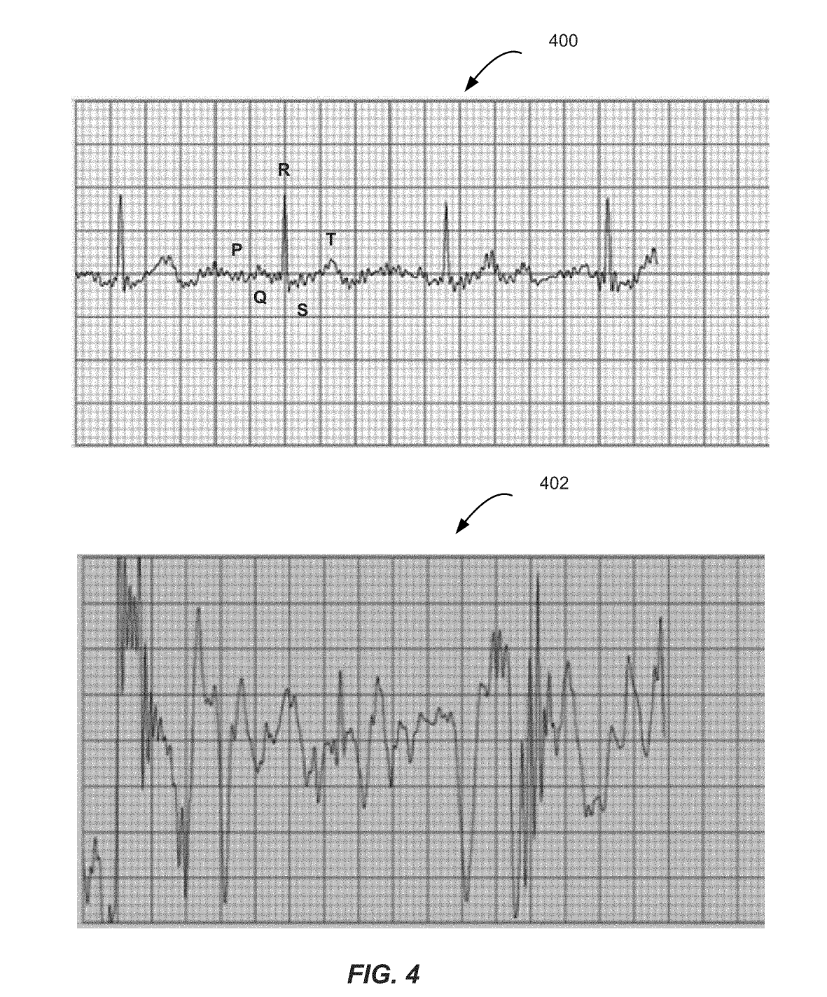

The data from sensors 114, 116 may be captured opportunistically, for example, as the user puts a palm or finger on the electrode 112 in proximity to or touching the proximity-sensitive surface 118 of the sensor 116. To ensure meaningful sensor readings, the sensor data, e.g., PPG readings by the sensor 116, may be captured over a determined period of time, for example, at least for five seconds. Optimum time for capturing sensor readings may be empirically configured for the apparatus 100. The captured sensor data may be time stamped as it is captured from the sensors. During the sensor readings processing, it may not be apparent whether the user touched the electrode 110, in addition to touching the electrode 112, and thus closed the circuit with the sensor 114 (ECG sensor). Accordingly, the captured ECG readings may be verified for validity by the integrated sensor hub 150 (e.g., when the processing block 120 is asleep or in stand-by mode), as described in reference to FIGS. 3-4. If the ECG readings are determined to be invalid (e.g., the user may have touched the electrode 112 and may not have touched the electrode 110), the processing of the ECG readings may be terminated.

The processing block 120 may comprise at least a processor 122 and memory 124. The processing block 120 may further include components configured to record and process the readings of the user's physiological context. The processing block 120 may provide these components through, for example, a plurality of machine-readable instructions stored in the memory 124 and executable on the processor 122.

The processor 122 may include, for example, one or more processors situated in separate components, or alternatively one or more processing cores embodied in a component (e.g., in an SoC configuration), and any processor-related support circuitry (e.g., bridging interfaces, etc.). Example processors may include, but are not limited to, various microprocessors including those in the Pentium®, Xeon®, Itanium®, Celeron®, Atom®, Quark®, Core® product families, or the like. Examples of support circuitry may include host side or input/output (I/O) side chipsets (also known as northbridge and southbridge chipsets/components) to provide an interface through which the processor 120 may interact with other system components that may be operating at different speeds, on different buses, etc. in apparatus 100. Some or all of the functionality commonly associated with the support circuitry may also be included in the same physical package as the processor.

The memory 124 may comprise random access memory (RAM) or read-only memory (ROM) in a fixed or removable format. RAM may include volatile memory configured to hold information during the operation of apparatus 100 such as, for example, static RAM (SRAM) or dynamic RAM (DRAM). ROM may include non-volatile (NV) memory circuitry configured based on basic input/output system (BIOS), Unified Extensible Firmware Interface (UEFI), etc. to provide instructions when apparatus 100 is activated, programmable memories such as electronic programmable ROMs (erasable programmable read-only memory), Flash, etc. Other fixed/removable memory associated with the apparatus 100 may include, but is not limited to, electronic memories such as solid state flash memory, removable memory cards or sticks, etc.

The integrated sensor hub 150 may be coupled with processor 122 and memory 124 and configured to aggregate and further process the data provided by sensors 114, 116, and other sensors 132 that may be included in the apparatus 100. In embodiments, the integrated sensor hub 150 may run autonomously, e.g., independent from processor 122 and memory 124 after booting up the processing block 120. The integrated sensor hub 150 may comprise a common low-power sensor hub, to allow opportunistic sensing whenever the user maintains direct (or indirect) contact between her hands (fingers, palms, fingers, wrists) and at least the electrode 112 with the proximity-sensitive surface 118. As shown, the integrated sensor hub 150 may be coupled with the sensors 114, 116, 132 via general purpose input/output (IO) circuit and/or via inter-integrated circuit I2C. Communication channels (IPC) may connect the integrated sensor hub 150 to other components in the SOC, such as the application processor (e.g., processor 122) and security engine (not shown). The integrated sensor hub 150 may be coupled with the processor 122 via communication fabric 160, and with the memory 124 via direct memory access (DMA) 162. In embodiments, sensor data acquisition (DAQ) and sensor fusion may be offloaded from the host to the integrated sensor hub 150, which may perform required sensor processing.

The processing block 120 may include other components necessary for functioning of the apparatus 100, some of which are not described herein for ease of understanding. For example, the processing block 120 may include a graphics processor GFX 126, and other components 130. Other components 130 may include, for example, one or more interfaces (not shown) to communicate the user's context measurements over one or more wired or wireless network(s) and/or with any other suitable device, such as external computing device (not shown). In embodiments, the processing of sensor readings may be performed by the integrated sensor hub 150's processor 152. In some embodiments, at least part of the processing may be performed by the processor 122.

The apparatus 100 may include other sensors 132 that may be coupled with the integrated sensor hub 150, as shown. Sensors 132 may comprise other types of sensors configured to measure the current physiological state of the user or other parameters. For example, sensors 132 may measure motions of the user in relation to the apparatus 100, jitter associated with user interaction with the apparatus 100 (e.g., user's interaction with a keyboard, touchscreen, or touchpad of the apparatus 100), user's body skin temperature, and the like. For example, sensors 132 may include one of accelerometer, gyroscope, temperature sensor, or the like.

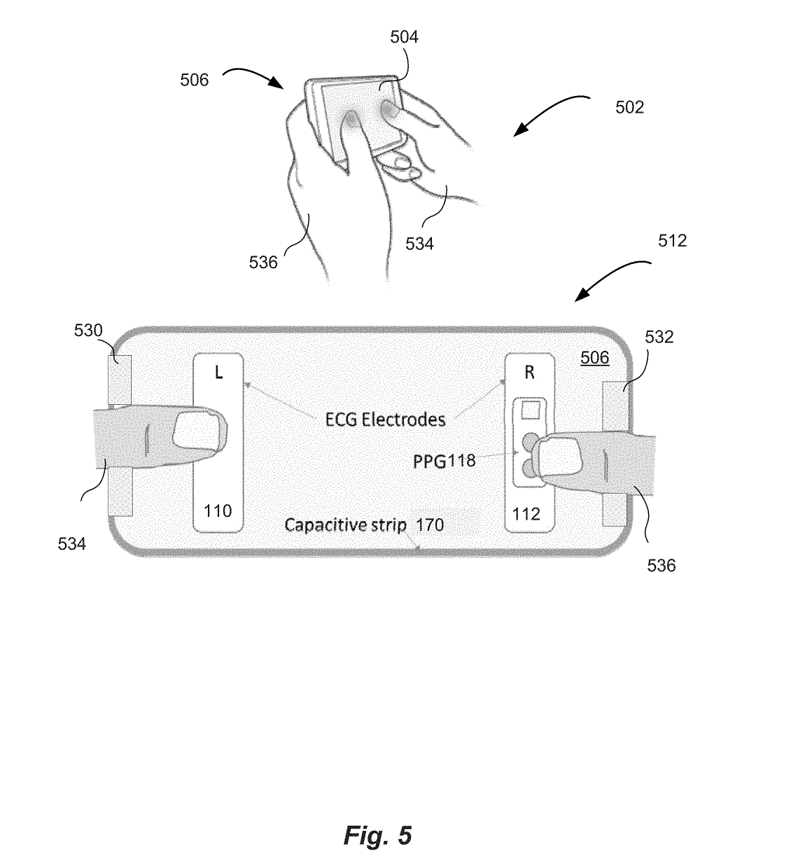

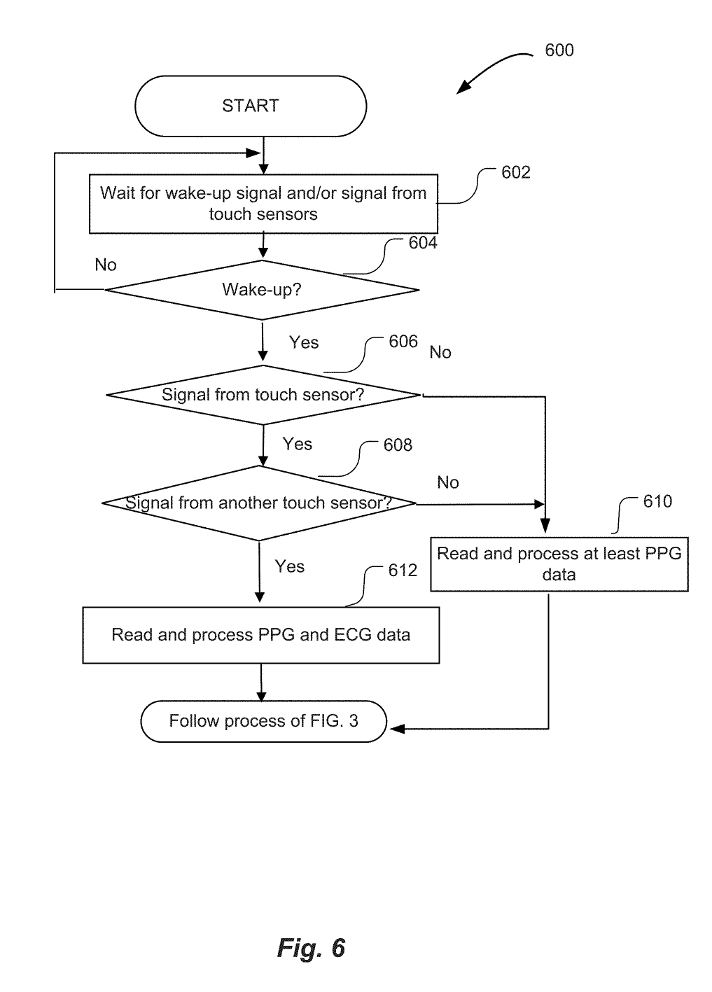

In some embodiments, the apparatus 100 may include touch sensors 170 (e.g., one or more capacitive strips) disposed about the work surface 102 of the apparatus 100, shown in dashed lines in FIG. 1. The touch sensors 170 may be configured to produce a signal 184 (shown in dashed line) in response to a detection of the user's touch of the work surface 102. The signal 184 may be combined with the wake-up signal 180 to control the provision of signal 180 to the integrated sensor hub 150. The embodiments of the apparatus 100 including the touch sensors 170 and its operation will be described in detail in reference to FIGS. 5-6.

The apparatus may further include circuitry configured to facilitate a provision of readings from different sensors embedded in or otherwise coupled with the apparatus 100. Such circuitry (not shown) may include, for example, an amplifier, an analog-to-digital converter (ADC) and a controller to operate the circuitry. In some embodiments, the circuitry may be integrated in a form of an integrated circuit (IC).

It should be noted that the number of electrodes and sensors illustrated and types of sensors provided are for illustration purposes only and are not to be construed as limiting on this disclosure.

The apparatus 100 may further include different components necessary for the functioning of the apparatus, depending on a type of the apparatus. For example, the apparatus 100 may include a camera, a flash, a microphone, and other components (not shown) that may be typically included in a computing or wearable device of a particular type. The apparatus 100 may further include a display (not shown) to display results of opportunistic measurements and processing of the user's physiological context.

As briefly described above, to allow for opportunistic sensing, electrodes 110, 112 may be accessible to the user in natural positions and activities involving the apparatus 100. There may be several options for placement of these sensors on devices. As described above, the apparatus 100 may include a work surface 102, such as a computing device keyboard, which may come in direct contact with the user's hands, palms, or wrists 106 when the user operates the keyboard. In another example, a computing device may comprise a tablet or smartphone, and work surfaces may comprise bezels or back sides of the respective devices. Some examples of sensor placement on work surfaces of various computing devices are described below.

FIG. 2 illustrates examples of disposition of sensors on work surfaces of computing devices, to enable measurements of a user's physiological context, in accordance with some embodiments. View 202 illustrates the placement of the electrodes around a bezel 204 of a casing 205 of a tablet computing device 206. View 212 illustrates the placement of the electrodes around a back side 208 of the casing 205 of a tablet computing device (e.g., 206). View 222 illustrates the placement of the electrodes on a keyboard 226 of a computing device, such as a laptop, tablet (if equipped with a keyboard), or desktop computer. For example, the electrodes may be disposed on no-key areas of the keyboard 226.

View 232 illustrates the placement of the electrodes around a work surface, such as back side 228 of a smartphone 236. As shown, the electrodes 110 (e.g., left electrode) and 112 (e.g., right electrode) may be dimensioned and disposed on the back side 228 to provide a high probability of an opportunistic contact with respective hands (e.g., fingers) of a user. As shown, the right electrode 112 may include the proximity-sensitive surface 118 of the PPG sensor (not shown), to enable detection of contact between the surface 118 and the user's right arm (finger), to trigger the wake-up signal to the processing block (not shown). As discussed above, the smartphone 236 may include other components, such as LED flash (which may be embedded within the electrode 112 for convenience, as shown), a camera, a microphone, and the like.

Accordingly, a computing device with the sensors for opportunistic measurements of the user context configured according to embodiments described herein may include a laptop computer, a desktop computer, a tablet computer, a smartphone, a wearable device, or any other mobile or stationary computing device. A work surface suitable for placing the sensors for measurements of a user's context may include at least a portion of a keyboard of a computing device, a bezel of the computing device, or a back side of the computing device.

It should be noted that the apparatus 100 may take a number of different forms, in addition or as an alternative to that described herein. For example, apparatus 100 may comprise, e.g., headsets, glasses, wands or styluses that contain compute components, and the like. Accordingly, different body parts (e.g., forehead, eyes, ears, etc.), in addition or in the alternative to arm portions, such as fingers, hands, palms or wrists, may be in direct or indirect contact with different forms of work surfaces of computing devices of different types, to enable the user's context measurements and processing described herein.

FIG. 3 is a process flow diagram for opportunistic measurements and processing of a user's physiological context, in accordance with some embodiments. The process 300 may comport with and be performed by some of the elements of the various embodiments earlier described in reference to FIGS. 1-2. In alternate embodiments, the process 300 may be practiced with more or fewer operations, or a different order of the operations. The process 300 may be performed, for example, by the processing block 120, such as integrated sensor hub 150 of the apparatus 100 of FIG. 1. Accordingly, the process 300 is described with continuous reference to FIG. 1.

The process 300 may begin at block 302 and include enabling and configuring sensors 114 and 116, and putting the sensors in a stand-by mode. The process of block 302 may further include waiting for a wake-up (e.g., “interrupt”) signal 180, which may be triggered in response to detecting of a touch (or proximity) of a portion of the user's arm (e.g., a finger) to the proximity-sensitive surface 118 of the sensor 116 (e.g., PPG sensor) embedded in the electrode 112 as described in reference to FIG. 1. Whenever the electrode 112 is touched (or proximity of the user's arm portion is detected), the sensor 116 (PPG sensor) may generate the wake-up signal 180, which may trigger the processing of PPG readings by the integrated sensor hub 150. The same wake-up signal 180 may be used as a “pseudo wake-up” signal to change the mode of the sensor 114 (ECG sensor) from stand-by mode to sense mode and invoke the related processing routine in the integrated sensor hub 150.

At decision block 304, the process 300 may include determining whether the wake-up signal has been received. If it is determined that the wake-up signal has not been received, the process 300 may return to block 302. If it is determined that the wake-up signal has been received, the readings and processing of the readings provided by sensors 114 and 116 (e.g., ECG and PPG sensors respectively) may commence. The processing of the ECG and PPG readings may occur independently and in parallel, as indicated by blocks 306 and 314 respectively.

Accordingly, at block 306, the process 300 may include reading and processing PPG data provided by the sensor 116. The processing block 120 (e.g., integrated sensor hub 150) may process the PPG data and extract heart rate and SpO2 data regardless of whether the electrode 110 is active (e.g., touched by the user).

At block 308, the process 300 may include storing the PPG data, e.g., in memory 124. The data may be stored with a time stamp, for example.

At decision block 310, the process 300 may include verifying whether the reading and processing of the PPG data may be completed. Different conditions may be met to satisfy the completion of the processing of the PPG data. For example, a particular time period (e.g., about 5 seconds) may be determined for the processing block to process the data. In another example, the wake-up signal 180 may be provided continuously for the duration of the user touching the proximity-sensitive surface 118. The wake-up signal 180 may terminate, in response the user moving a portion of her arm (e.g., a finger) away from the proximity-sensitive surface 118. If any of these conditions occur, the processing of the PPG data may be completed at block 312, and the process 300 may return to block 302. Otherwise, the process 300 may return to block 306. At block 314, the process 300 may include reading and processing ECG data provided by the sensor 114. As noted above, the process of block 314 may occur in parallel to the processes described in blocks 306, 308, and 310.

At decision block 316, the process 300 may include determining whether the ECG data provided by the sensor 114 is valid. For example, the processing block 120 may activate the ECG sensor and read the ensuing packet header information of the ECG data provided by sensor 114 to determine if the ECG data is valid, e.g., when the data is generated when both electrodes 110, 112 are touched by the user's respective portions of hands (e.g., left and right fingers respectively). The ECG data validation is described in reference to FIG. 4.

If the ECG data is determined to be invalid, e.g., electrode 110 is not touched by the user's arm substantially simultaneously with the user's other arm touching the electrode 112, the reading and processing of the ECG may terminate at block 318, and the process 300 may return to block 302. In another example, the processing block 120 may enter a polling mode for the duration of the PPG data reading and processing. This may be done in a case where the user may initially touch the electrode 112 only with proximity-sensitive surface 118, activating the PPG processing, and (e.g., later in time) may touch the electrode 110 after the wake-up signal 180 is no longer active.

If at decision block 316 the ECG data is determined to be valid, the process 300 may move to block 320, which may include storing the ECG data, e.g., in memory 154, to avoid accessing the host system memory unnecessarily in order to save power. The data may be stored with a time stamp, for example.

At decision block 322 it may be determined whether the reading and processing of the ECG data may be terminated. The termination of the readings may be done, for example, if the time period allocated for ECG data reading and processing may have expired. In another example, the wake-up signal may be de-asserted (e.g., the user removes her finger from the electrode 112, as described above). In some instances, it may be possible for the user to remove their finger from electrode 110 mid-stream (after a few seconds) while still touching electrode 112. In this case, the ECG may no longer be valid. This case may be detected in the integrated sensor hub by, for example, monitoring the integrity of the inter beat interval (IBI) data and terminating the processing when this data is invalid, such as when the data falls outside expected limits. If it is determined that the reading and processing may not be terminated, the process 300 may return to block 314. If it is determined that the reading and processing may be terminated, the process 300 may move to block 318, at which the reading and processing of the ECG data may be terminated.

In summary, because the ECG sensor may not have means of reporting a closed loop analog front end (AFE) condition, the integrated sensor hub 150 may read the ECG output data, unpack the header packets, and process the data to determine ECG signal validity. If the data is deemed valid, the ECG data stream may be read and processed until one of two conditions is manifested: the wake-up signal is de-asserted (e.g., the user removes her finger from the electrode 112) or the ECG data is no longer valid (e.g., the user removes her finger from the electrode 110).

FIG. 4 illustrates example graphs of ECG data, in accordance with some embodiments. The graphs illustrate ECG data validation techniques briefly described in reference to FIG. 3. More specifically, graph 400 illustrates valid ECG data that may be read from the ECG sensor, e.g., sensor 114, and graph 402 illustrates invalid ECG data that may be read from the ECG sensor. In order to determine whether ECG data is valid, an R-R peak detection algorithm may be used to capture a few, e.g., three to five R peaks of a typical PQRST complex of an ECG graph (as shown in the graph 400). A PQRST complex of an ECG signal may include three waves. Q-wave may indicate the downward deflection of the ECG signal. R-wave may indicate the upward deflection from point Q to point R. S-wave may indicate the downward deflection from point R to point S. A P-wave may occur before the QRS complex and a T-wave may follow the QRS complex. Accordingly, the time intervals between the peaks of the PQRST complex may be analyzed and it may be determined whether the waves are representative of typical ECG waveforms. For example, if the time intervals between the peaks of the PQRST complex (e.g., IBI) fall within respective typical ranges, it may be inferred that the ECG data represented by the PQRST complex is valid. In another example, a single PQRST complex (e.g., about 200 data samples) may be analyzed to determine validity of the ECG data.

As discussed in reference to FIG. 1, in some embodiments, the apparatus 100 may include touch sensors 170 (e.g., one or more capacitive strips or other touch-sensitive surfaces) disposed about the work surface 102 of the apparatus 100 (e.g., around the bezel of the apparatus 100). The touch sensors 170 may be configured to produce a signal 184 in response to a detection of the user's touch of the work surface 102. The signal 184 may be combined with the wake-up signal 180 to control the provision of signal 180 to the processing block 120. The touch sensors may detect contact between the work surface 102 of the apparatus 100 and the at least portions of respective hands of a user. The processing block 102 may begin processing the ECG and PPG readings in response to the receipt of the wake-up signal 180 and further in response to a receipt of the signal 184, indicating the detection of contact between the work surface 102 and the at least a portion of an arm (or portions of respective arms) of the user.

FIG. 5 illustrates an example embodiment of an apparatus for opportunistic measurements of the user's physiological context, suitable for use with various components of FIG. 1, in accordance with some embodiments. More specifically, the apparatus 100 (e.g., a smartphone) is shown in perspective view 502 and in back view 512. While the embodiments of FIG. 5 are described in smartphone implementation, other embodiments are also possible, for example, with respect to devices described in reference to FIG. 2, such as laptop, tablet computer, and the like.

As shown in view 502, the user may hold a smartphone 504 with a work surface (e.g., back side) 506 with parts of the user's respective hands 534, 536. The back side 506 of the smartphone 504 is shown in view 512. As shown, the touch sensors 170 referenced in FIG. 1, such as one or more capacitive touch-sensitive surfaces or pressure sensors, may be disposed around the back side 506 (e.g., around the edge of the smartphone 504). Although shown as one continuous strip in view 512, the touch sensors 170 may comprise several (e.g., two or more) discrete portions or sections (e.g., segments 530 and 532) making it possible to determine whether the user is holding the device with left, right, or both left and right hands. For example, if segment 530 is active by the user's finger 534 touching it, there is a probability that electrode 110 may also be active and ECG data reading and processing may commence.

In the event the user is holding the device with both hands, it may be inferred that there is at least a possibility that both electrodes 110 and 112 may be touched by the user. Further, the proximity-sensitive surface 118 may report, in addition to the report by sensor 170, that the user is touching the electrode 112. Accordingly, both PPG and EKG sensors may be turned on and the respective readings and processing may commence according to the process described in reference to FIG. 3. This arrangement provides means of identifying a probability that both left and right electrodes have been touched by the user.

FIG. 6 is a process flow diagram for opportunistic measurements and processing of the user's physiological context by the apparatus configured as described in reference to FIGS. 1 and 5, in accordance with some embodiments. The process 600 may be performed, for example, by the integrated hub 150 of the apparatus 100 of FIG. 1. In the example described by process 600, it is assumed that the touch sensors 170 may include two discrete portions 530 and 532, which may allow detection of the touch of the work surface 102 (e.g., back side 506) of the apparatus 100 (e.g., smartphone 504).

The process 600 may begin at block 602, and include waiting for a wake-up signal, as described in reference to block 302 of FIG. 3. The process at block 602 may further include waiting for an indication from at least one of the touch sensor portions 530 or 532 that the user touched the back surface 506.

At decision block 604 it may be determined whether the wake-up signal has been received.

If the wake-up signal has been received, at decision block 606 it may be determined whether a signal from at least one touch sensor (e.g., 530 or 532) has been received.

If no signal from the touch sensor has been received, the process 600 may move to block 610, where the reading and processing of at least PPG data may commence, in response to a receipt of the wake-up signal provided by the touch-sensitive surface of the PPG sensor.

If a signal from the touch sensor has been received, at decision block 608 it may be determined whether a signal from another touch sensor has been received.

If no signal from another touch sensor has been received, the process 600 may move to block 610, where the reading and processing of at least PPG data may commence. If additionally it may be determined that, for example, the signal is received from a touch sensor that corresponds to a portion of the left hand 534 of the user, it may be inferred that there is a probability that the user may touch the left electrode 110 (with reference to FIG. 5). Accordingly, in addition to reading and processing of the PPG data, the reading and processing of the ECG data may also commence.

If a signal from another touch sensor has been received, it may be understood that the user is touching the back side 506 with portions of both hands 534, 536. Thus, in addition to touching the right electrode 112 with the proximity-sensitive surface 118 with the user's right hand 536, as indicated by the wake-up signal, the user may be touching the left electrode 110, at least with some probability. It may be assumed that the ECG data may be read and processed. Accordingly, the process 600 may move to block 612, where the reading and processing of PPG and ECG data may commence.

The process 600 may then move from blocks 610 or 612 to the process described in reference to FIG. 3, namely, to the ECG data validation (if desired), and the determinations whether the readings of ECG and/or PPG data may be terminated.

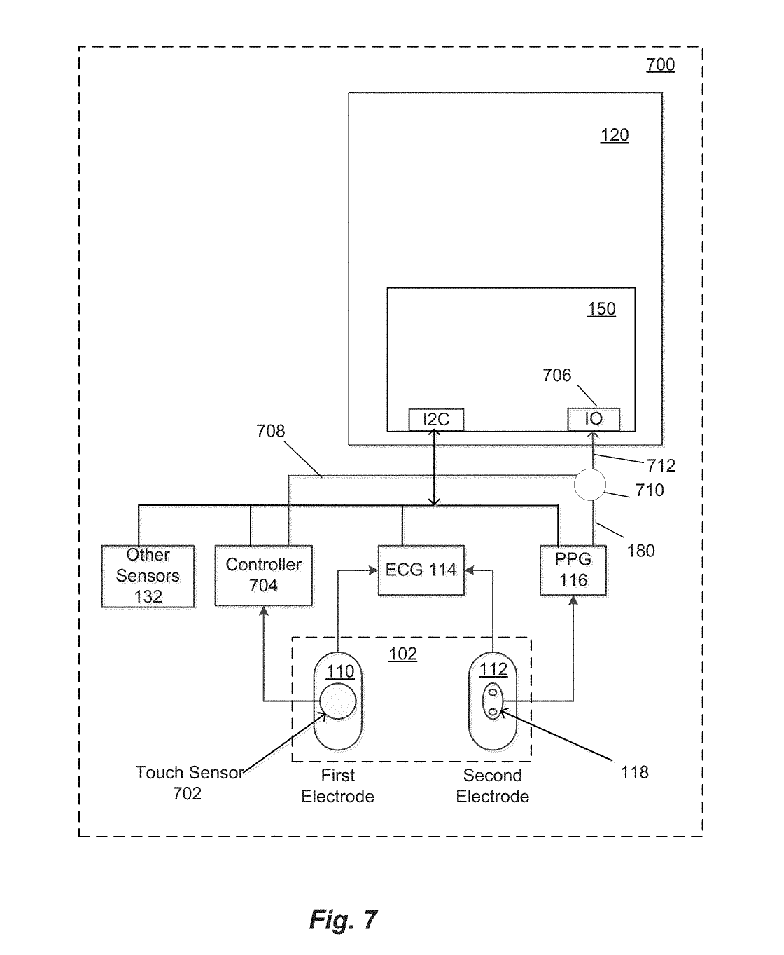

FIG. 7 is a block diagram illustrating an example apparatus for opportunistic measurements of a user's physiological context, in accordance with some embodiments. At least some of the components of apparatus 700 are similar to those of the apparatus 100 of FIG. 1 and are indicated by like numerals, for ease of understanding. For ease of understanding and brevity, the descriptions of the like components of FIGS. 1 and 7 are omitted.

As shown, a touch-sensitive sensor 702 (e.g., capacitive touch surface or a pressure sensor) may be embedded in the electrode 110 of the apparatus 700, in addition to the proximity-sensitive surface 118 of the sensor 116 that may be embedded in the electrode 112. The touch-sensitive sensor 702 may be coupled with a touch sensor controller 704. The touch-sensitive sensor 702 may be configured to sense a touch by the user's arm portion (e.g., palm or finger) of the electrode 110, and the sensor controller may generate a corresponding wake-up signal 708. The described arrangement may provide for determining when both electrodes 110 and 112 may have been touched by the user's respective arm portions.

In order to preserve the use of a single general purpose IO input 706 (e.g., pin), as shown in the diagram, the wake-up signal 708 from the controller 704 and the wake-up signal 180 of the sensor 116 (PPG sensor) may be combined together in an OR or AND combination at a gate 710, to generate a wake-up signal 712 for the integrated sensor hub 150. More specifically, the integrated sensor hub 150 may access the status registers of the PPG controller (integrated with the sensor 116) and the touch sensor controller 704 to ascertain which of the sensors 702 and/or 116 may be active. For example, if ECG and PPG signals may need to be monitored simultaneously, the signals 708 and 180 may be combined in an AND combination, before being fed to the GPIO pin 706 in a form of a wake-up signal 712, to trigger the servicing of both PPG and ECG sensors simultaneously.

If the ECG and PPG signals are to be asynchronously or independently monitored and processed, the gate 710 may be an OR gate. Then, the signals 708 and 180 may be combined in an OR combination and then fed to the GPIO pin 706, to provide the signal 712, to wake up the integrated sensor hub 150.

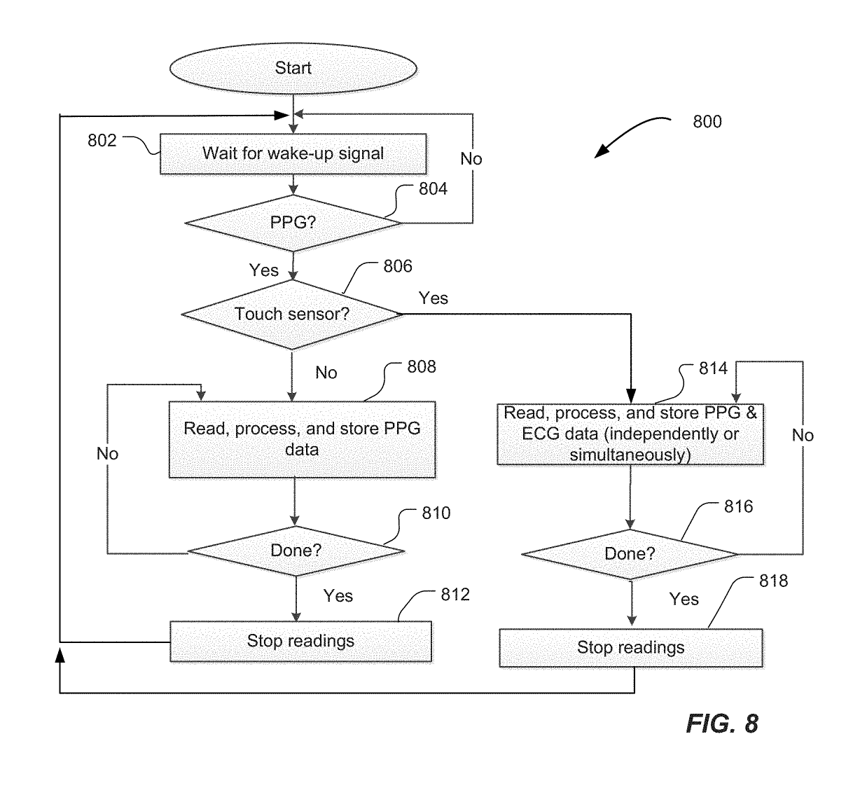

FIG. 8 is a process flow diagram for opportunistic measurements and processing of a user's physiological context with an apparatus of FIG. 7, in accordance with some embodiments. The process 800 may comport with and be performed by some of the elements of the various embodiments earlier described in reference to FIGS. 1 and 7. In alternate embodiments, the process 800 may be practiced with more or fewer operations, or different order of the operations. The process 800 may be performed, for example, by the integrated sensor hub 150 of the apparatus 700 of FIG. 7. Accordingly, the process 800 is described with continuous reference to FIGS. 1 and 7. Some of the operations of the process 800 may be performed similar to the like-named operations of the process 300 of FIG. 3. The descriptions of such operations are omitted for brevity.

The process 800 may start at block 802 and include waiting for a wake-up signal 712. Referencing FIG. 7, signal 712 may be triggered by the signal 180 generated by the PPG sensor 112 in response to detection of proximity of the user's hand by the proximity-sensitive surface 118 to the gate 710, or by signal 708 provided by the controller 704 in response to a detection of touch by the touch sensor 702.

At decision block 804, the process 800 may include determining whether the wake-up signal 712 resulted from the signal 180 from the PPG sensor 116. If it is determined that the wake-up signal 712 was not triggered by PPG signal 180, the process 800 may return to block 802.

If it is determined that the wake-up signal 712 has been triggered by PPG sensor (signal 180), at decision block 806 it may be determined whether the wake-up signal 708 from the controller 704 has also been generated. As noted, the signal 708 from the touch sensor 702 may be generated in response to the user touching the touch sensor 702.

If it is determined that the signal 708 has not been generated by the sensor 702, at block 808 the PPG data may be read, processed, and stored, similar to operations described in reference to FIG. 3. At decision block 810, the process 800 may include verifying whether the reading and processing of the PPG data may be terminated, similar to operations described in reference to FIG. 3. If it is determined that the reading and processing may be terminated, the processing of the PPG data may be terminated at block 812, and the process 800 may return to block 802. Otherwise, the process 800 may return to block 808.

If at decision block 806 it is determined that the signal 708 has been generated, at block 814 the ECG and PPG data may be read, processed, and stored, similar to the operations described in reference to FIG. 3. For example, the ECG and PPG data may be processed simultaneously if the signals 708 and 180 may be combined in an AND combination, as discussed in reference to FIG. 7. In another example, the ECG and PPG data may be processed asynchronously or independently if the signals 708 and 180 may be combined in an OR combination, as discussed in reference to FIG. 7. At decision block 816 it may be determined whether the readings of PPG and ECG data may be terminated, similar to operations described in reference to FIG. 3. If it is determined that the reading and processing may be terminated, the processing of the PPG and ECG data may be terminated at block 818, and the process 800 may return to block 802. Otherwise, the process 800 may return to block 814.

The embodiments described herein may be further illustrated by the following examples.

Example 1 may be an apparatus for providing a user's physiological context, comprising: a processing block; a first sensor coupled with the processing block and having first and second electrodes disposed on a work surface of the apparatus, to provide first readings of a user's physiological context in response to a contact between the first and second electrodes and at least portions of respective first and second hands of a user during interaction of the user with the apparatus; and a second sensor coupled with the processing block and having a sensitive surface embedded in one of the first or second electrode of the first sensor, wherein the second sensor is to provide second readings of the user's physiological context and to further provide a wake-up signal to the processing block in response to at least a proximity of a portion of one of the first or second hands to the sensitive surface, wherein the processing block is to facilitate processing of the user's physiological context in response to a receipt of the wake-up signal.

Example 2 may include the subject matter of Example 1, wherein the first sensor comprises an electrocardiogram (ECG) sensor, wherein the first readings include ECG data, wherein the second sensor comprises a photoplethysmogram (PPG) sensor, wherein the second readings include PPG data.

Example 3 may include the subject matter of Example 1, wherein the processing block is to begin processing the first readings in response to the receipt of the wake-up signal.

Example 4 may include the subject matter of Example 3, wherein the processing block is to stop processing the first readings, in response to a termination of the receipt of the wake-up signal.

Example 5 may include the subject matter of Example 3, wherein the processing block is to: determine whether the first readings are valid; and stop processing the first readings or periodically poll the first sensor in response to a determination that the first readings are not valid.

Example 6 may include the subject matter of Example 5, wherein the processing block is to begin processing the second readings in response to the receipt of the wake-up signal.

Example 7 may include the subject matter of Example 6, wherein the processing block is to stop processing the second readings in response to a determination that the second readings have been collected.

Example 8 may include the subject matter of Example 1, wherein the first sensor includes a first sensitive surface embedded in the first electrode, wherein the sensitive surface of the second sensor is a second sensitive surface, wherein the one of the first or second electrode is the second electrode, wherein the one of the first or second hands is the first hand, wherein the second sensor is to provide the wake-up signal to the processing block in further response to a contact between the second hand and the first sensitive surface.

Example 9 may include the subject matter of Example 8, wherein the processing block is to begin processing the first or second readings in response to the receipt of the wake-up signal.

Example 10 may include the subject matter of Example 9, wherein the processing block is to stop processing the first and second readings in response to a termination of the receipt of the wake-up signal.

Example 11 may include the subject matter of Example 1, wherein the processing block is integrated on a system on chip (SOC), wherein the apparatus comprises one of: a laptop computer, a desktop computer, a tablet computer, a smartphone, a set-top box, a game controller, or a wearable device.

Example 12 may include the subject matter of Example 11, wherein the processing block includes an integrated sensor hub.

Example 13 may include the subject matter of Example 11, wherein the work surface comprises at least a selected one of: a keyboard of the apparatus, a bezel of the apparatus, or a back side of the apparatus, wherein the portions of first and second hands include at least one of: wrists, palms, hands, or fingers that are disposed on the work surface to interact with the apparatus.

Example 14 may include the subject matter of any Examples 1 to 13, further comprising third and fourth sensors disposed around the work surface, to detect a contact between the work surface and the at least portions of respective first and second hands of a user, wherein the processing block is to begin processing the first readings and second readings in response to the receipt of the wake-up signal and a receipt of an indication of the detection of contact between the work surface and the at least portions of respective first and second hands.

Example 15 may include the subject matter of Example 14, wherein the processing block is to: determine whether the first readings are valid; and stop processing the first readings in response to a determination that the first readings are not valid.

Example 16 may be a computing device-implemented method for providing a user's physiological context, comprising: obtaining, by a computing device communicatively coupled with first and second sensors disposed in an apparatus that includes the computing device, a wake-up signal initiated in response to at least a proximity of a portion of one of the first or second hands to a sensitive surface of the second sensor, the sensitive surface embedded in one of a first or second electrode coupled with the first sensor and disposed on a work surface of the apparatus, wherein the first sensor is to provide first readings of a user's physiological context in response to a contact between the first and second electrodes and at least portions of respective first and second hands of a user during interaction of the user with the apparatus, herein the second sensor is to provide second readings of the user's physiological context; and initiating, by the computing device, a processing of the user's physiological context in response to obtaining the wake-up signal, including processing of at least the second readings, and determining whether to process the first readings.

Example 17 may include the subject matter of Example 16, further comprising: determining, by the computing device, whether the first readings are valid; and terminating the processing of the first readings or initiating a periodic polling of the first sensor, by the computing device, in response to a determination that the first readings are not valid.

Example 18 may include the subject matter of Example 16, wherein the first sensor includes a first sensitive surface embedded in the first electrode, wherein the sensitive surface of the second sensor is a second sensitive surface, wherein the one of the first or second electrode is the second electrode, wherein the one of the first or second hands is the first hand, wherein obtaining a wake-up signal includes receiving, by the computing device, the wake-up signal from the second sensor in further response to a contact between the second hand and the first sensitive surface.

Example 19 may include the subject matter of Example 18, wherein initiating a processing of the user's physiological context in response to obtaining the wake-up signal includes: processing, by the computing device, the first and second readings.

Example 20 may include the subject matter of any Examples 16 to 19, wherein the apparatus includes third and fourth sensors disposed around the work surface, to detect a contact between the work surface and the at least portions of respective first and second hands of a user, wherein initiating a processing of the user's physiological context in response to obtaining the wake-up signal includes: processing, by the computing device, the first and second readings in response to the receipt of the wake-up signal and a receipt of an indication of the detection of contact between the work surface and the at least portions of respective first and second hands.

Example 21 may be one or more non-transitory computing device-readable media having executable instructions for providing a user's physiological context stored thereon that, in response to execution, cause a computing device communicatively coupled with first and second sensors disposed in an apparatus that includes the computing device, to: obtain a wake-up signal initiated in response to at least a proximity of a portion of one of the first or second hands to a sensitive surface of the second sensor, the sensitive surface embedded in one of a first or second electrode coupled with the first sensor and disposed on a work surface of the apparatus, wherein the first sensor is to provide first readings of a user's physiological context in response to a contact between the first and second electrodes and at least portions of respective first and second hands of a user during interaction of the user with the apparatus, wherein the second sensor is to provide second readings of the user's physiological context; and initiate a processing of the user's physiological context in response to obtaining the wake-up signal, wherein to initiate includes process at least the second readings, and determine whether to process the first readings.

Example 22 may include the subject matter of Example 21, wherein the instructions further cause the computing device to: determine whether the first readings are valid; and terminate the processing of the first readings or initiate a periodic polling of the first sensor, by the computing device, in response to a determination that the first readings are not valid.

Example 23 may include the subject matter of Example 21, wherein the first sensor includes a first sensitive surface embedded in the first electrode, wherein the sensitive surface of the second sensor is a second sensitive surface, wherein the one of the first or second electrode is the second electrode, wherein the one of the first or second hands is the first hand, wherein the second sensor is to provide the wake-up signal to the processing block in further response to a contact between the second hand and the first sensitive surface.

Example 24 may include the subject matter of Example 23, wherein the instructions to initiate a processing of the user's physiological context in response to obtaining the wake-up signal cause the computing device to process the first and second readings.

Example 25 may include the subject matter of any Examples 21 to 24, wherein the apparatus includes third and fourth sensors disposed around the work surface, to detect a contact between the work surface and the at least portions of respective first and second hands of a user, wherein the instructions to initiate a processing of the user's physiological context cause the computing device to process the first and second readings in response to the receipt of the wake-up signal and a receipt of an indication of the detection of contact between the work surface and the at least portions of respective first and second hands.

Example 26 may be an apparatus having first or second sensors disposed in the apparatus for providing a user's physiological context, wherein the apparatus comprises: means for obtaining a wake-up signal initiated in response to at least a proximity of a portion of one of the first or second hands to a sensitive surface of the second sensor, the sensitive surface embedded in one of a first or second electrode coupled with the first sensor and disposed on a work surface of the apparatus, wherein the first sensor is to provide first readings of a user's physiological context in response to a contact between the first and second electrodes and at least portions of respective first and second hands of a user during interaction of the user with the apparatus, wherein the second sensor is to provide second readings of the user's physiological context; and means for initiating a processing of the user's physiological context in response to obtaining the wake-up signal, including processing of at least the second readings, and determining whether to process the first readings.

Example 27 may include the subject matter of Example 26, further comprising: means for determining whether the first readings are valid; and means for terminating the processing of the first readings or initiating a periodic polling of the first sensor in response to a determination that the first readings are not valid.

Example 28 may include the subject matter of Example 26, wherein the first sensor includes a first sensitive surface embedded in the first electrode, wherein the sensitive surface of the second sensor is a second sensitive surface, wherein the one of the first or second electrode is the second electrode, wherein the one of the first or second hands is the first hand, wherein means for obtaining a wake-up signal includes means for receiving the wake-up signal from the second sensor in further response to a contact between the second hand and the first sensitive surface.

Example 29 may include the subject matter of Example 28, wherein means for initiating a processing of the user's physiological context in response to obtaining the wake-up signal includes means for processing the first and second readings.

Example 30 may include the subject matter of any Examples 26 to 29, wherein the apparatus includes third and fourth sensors disposed around the work surface, to detect a contact between the work surface and the at least portions of respective first and second hands of a user, wherein means for initiating a processing of the user's physiological context in response contact between the work surface and the at least portions of respective first and second hands.to obtaining the wake-up signal includes means for processing the first and second readings in response to the receipt of the wake-up signal and a receipt of an indication of the detection of

Various operations are described as multiple discrete operations in turn, in a manner that is most helpful in understanding the claimed subject matter. However, the order of description should not be construed as to imply that these operations are necessarily order dependent. Embodiments of the present disclosure may be implemented into a system using any suitable hardware and/or software to configure as desired.

Although certain embodiments have been illustrated and described herein for purposes of description, a wide variety of alternate and/or equivalent embodiments or implementations calculated to achieve the same purposes may be substituted for the embodiments shown and described without departing from the scope of the present disclosure. This application is intended to cover any adaptations or variations of the embodiments discussed herein. Therefore, it is manifestly intended that embodiments described herein be limited only by the claims and the equivalents thereof.

What is claimed is:

1. An apparatus for providing a user's physiological context, comprising:
a processing block;
a first sensor coupled with the processing block and having first and second electrodes disposed on a work surface of the apparatus, to provide first readings of a user's physiological context in response to a contact between the first and second electrodes and at least portions of respective first and second hands of a user during interaction of the user with the apparatus; and
a second sensor coupled with the processing block and having a sensitive surface embedded in one of the first or second electrode of the first sensor, wherein the second sensor is to provide second readings of the user's physiological context and to further provide a wake-up signal to the processing block in response to at least proximity of a portion of one of the first or second hands to the sensitive surface, wherein the processing block is to facilitate processing of the user's physiological context in response to a receipt of the wake-up signal.

2. The apparatus of claim 1, wherein the first sensor comprises an electrocardiogram (ECG) sensor, wherein the first readings include ECG data, wherein the second sensor comprises a photoplethysmogram (PPG) sensor, wherein the second readings include PPG data.

3. The apparatus of claim 1, wherein the processing block is to begin processing the first readings in response to the receipt of the wake-up signal.

4. The apparatus of claim 3, wherein the processing block is to stop processing the first readings, in response to a termination of the receipt of the wake-up signal.

5. The apparatus of claim 3, wherein the processing block is to:
determine whether the first readings are valid; and
stop processing the first readings or periodically poll the first sensor in response to a determination that the first readings are not valid.

6. The apparatus of claim 5, wherein the processing block is to begin processing the second readings in response to the receipt of the wake-up signal.

7. The apparatus of claim 6, wherein the processing block is to stop processing the second readings in response to a determination that the second readings have been collected.

8. The apparatus of claim 1, wherein the first sensor includes a first sensitive surface embedded in the first electrode, wherein the sensitive surface of the second sensor is a second sensitive surface, wherein the one of the first or second electrode is the second electrode, wherein the one of the first or second hands is the first hand, wherein the second sensor is to provide the wake-up signal to the processing block in further response to a contact between the second hand and the first sensitive surface.

9. The apparatus of claim 8, wherein the processing block is to begin processing the first or second readings in response to the receipt of the wake-up signal.

10. The apparatus of claim 9, wherein the processing block is to stop processing the first and second readings in response to a termination of the receipt of the wake-up signal.

11. The apparatus of claim 1, wherein the processing block is integrated on a system on chip (SOC), wherein the apparatus comprises one of: a laptop computer, a desktop computer, a tablet computer, a smartphone, a set-top box, a game controller, or a wearable device.

12. The apparatus of claim 11, wherein the processing block includes an integrated sensor hub.

13. The apparatus of claim 11, wherein the work surface comprises at least a selected one of: a keyboard of the apparatus, a bezel of the apparatus, or a back side of the apparatus, wherein the portions of first and second hands include at least one of: wrists, palms, hands, or fingers that are disposed on the work surface to interact with the apparatus.

14. The apparatus of claim 1, further comprising third and fourth sensors disposed around the work surface, to detect a contact between the work surface and the at least portions of respective first and second hands of a user, wherein the processing block is to begin processing the first readings and second readings in response to the receipt of the wake-up signal and a receipt of an indication of the detection of contact between the work surface and the at least portions of respective first and second hands.

15. The apparatus of claim 14, wherein the processing block is to:
determine whether the first readings are valid; and
stop processing the first readings in response to a determination that the first readings are not valid.

16. A computing device-implemented method, comprising:
obtaining, by a computing device communicatively coupled with first and second sensors disposed in an apparatus that includes the computing device, a wake-up signal initiated in response to at least proximity of a portion of one of the first or second hands to a sensitive surface of the second sensor, the sensitive surface embedded in one of a first or second electrode coupled with the first sensor and disposed on a work surface of the apparatus, wherein the first sensor is to provide first readings of a user's physiological context in response to a contact between the first and second electrodes and at least portions of respective first and second hands of a user during interaction of the user with the apparatus, wherein the second sensor is to provide second readings of the user's physiological context; and
initiating, by the computing device, a processing of the user's physiological context in response to obtaining the wake-up signal, including processing of at least the second readings, and determining whether to process the first readings.

17. The computing device-implemented method of claim 16, further comprising:
determining, by the computing device, whether the first readings are valid; and
terminating the processing of the first readings or initiating a periodic polling of the first sensor, by the computing device, in response to a determination that the first readings are not valid.

18. The computing device-implemented method of claim 16, wherein the first sensor includes a first sensitive surface embedded in the first electrode, wherein the sensitive surface of the second sensor is a second sensitive surface, wherein the one of the first or second electrode is the second electrode, wherein the one of the first or second hands is the first hand, wherein obtaining a wake-up signal includes receiving, by the computing device, the wake-up signal from the second sensor in further response to a contact between the second hand and the first sensitive surface.

19. The computing device-implemented method of claim 18, wherein initiating a processing of the user's physiological context in response to obtaining the wake-up signal includes: processing, by the computing device, the first and second readings.

20. The computing device-implemented method of claim 16, wherein the apparatus includes third and fourth sensors disposed around the work surface, to detect a contact between the work surface and the at least portions of respective first and second hands of a user,
wherein initiating a processing of the user's physiological context in response to obtaining the wake-up signal includes: processing, by the computing device, the first and second readings in response to the receipt of the wake-up signal and a receipt of an indication of the detection of contact between the work surface and the at least portions of respective first and second hands.

21. One or more non-transitory computing device-readable media having executable instructions stored thereon that, in response to execution, cause a computing device communicatively coupled with first and second sensors disposed in an apparatus that includes the computing device, to:
obtain a wake-up signal initiated in response to at least proximity of a portion of one of the first or second hands to a sensitive surface of the second sensor, the sensitive surface embedded in one of a first or second electrode coupled with the first sensor and disposed on a work surface of the apparatus, wherein the first sensor is to provide first readings of a user's physiological context in response to a contact between the first and second electrodes and at least portions of respective first and second hands of a user during interaction of the user with the apparatus, wherein the second sensor is to provide second readings of the user's physiological context; and
initiate a processing of the user's physiological context in response to obtaining the wake-up signal, wherein to initiate includes process at least the second readings, and determine whether to process the first readings.

22. The non-transitory computing device-readable media of claim 21, wherein the instructions further cause the computing device to:
determine whether the first readings are valid; and
terminate the processing of the first readings or initiate a periodic polling of the first sensor, by the computing device, in response to a determination that the first readings are not valid.

23. The non-transitory computing device-readable media of claim 21, wherein the first sensor includes a first sensitive surface embedded in the first electrode, wherein the sensitive surface of the second sensor is a second sensitive surface, wherein the one of the first or second electrode is the second electrode, wherein the one of the first or second hands is the first hand, wherein the second sensor is to provide the wake-up signal to the processing block in further response to a contact between the second hand and the first sensitive surface.

24. The non-transitory computing device-readable media of claim 23, wherein the instructions to initiate a processing of the user's physiological context in response to obtaining the wake-up signal cause the computing device to process the first and second readings.

25. The non-transitory computing device-readable media of claim 21, wherein the apparatus includes third and fourth sensors disposed around the work surface, to detect a contact between the work surface and the at least portions of respective first and second hands of a user,
wherein the instructions to initiate a processing of the user's physiological context cause the computing device to process the first and second readings in response to the receipt of the wake-up signal and a receipt of an indication of the detection of contact between the work surface and the at least portions of respective first and second hands.
